(19) United States
(12) Patent Application Publication
(10) Pub. No.: US 2025/0151714 A1
(43) Pub. Date: May 15, 2025

(54) Methods for Improving Tissue Graft Storage Conditions through the Reduction of Senescence

(71) Applicant: Restoration Biologics LLC, Flagstaff, AZ (US)

(72) Inventors: Suzanne TABBAA, Dublin, OH (US); Sealy HAMBRIGHT, San Angelo, TX (US); Jeremy MALIK, San Diego, CA (US)

(21) Appl. No.: 18/946,035

(22) Filed: Nov. 13, 2024

Related U.S. Application Data
(60) Provisional application No. 63/598,336, filed on Nov. 13, 2023.

(51) Int. Cl. A01N 1/122; C12N 5/00

(52) U.S. Cl. CPC A01N 1/122; C12N 5/0081

(57) ABSTRACT

A method of removing senescent cells from donor tissues or cells, organs, or autologous cells or tissues forms a tissue graft. The method comprises adding at least one senolytic therapy to the tissue or cells during processing or prior to storage. The senolytic therapy preferably involves any strategy to reduce senescent cells, reduce senescence phenotype, reduce factors that can cause senescent cells, target known senescence signaling (e.g. inhibitors or RNA therapeutics). The viable tissue graft comprises live tissue with enhanced biological properties. The amount of senescent cells in the graft is reduced by at least 15% as compared to an amount of senescent cells present in normal tissue. The amount of senescence-associated secretory phenotype SASPs in the graft is reduced by at least 10% as compared to an amount of senescence-associated secretory phenotype SASPs present in normal cells.

### CROSS REFERENCE TO RELATED APPLICATIONS

This application claims the benefit of U.S. Provisional Application No. 63/598,336 titled “Methods for Improving Tissue Graft Storage Conditions Through the Reduction of Senescence” and filed on Nov. 13, 2023, which is incorporated herein by reference.

### FIELD OF THE INVENTION

The present disclosure relates to the use of senolytic agents or senolytic therapy approaches to clear senescent cells and improve cell health in donor tissue or organs containing live cells or autologous cells and tissues. The disclosure further relates to methods for improving outcomes following allograft tissue implantation. The disclosure further relates to methods for improving shelf-life or health of the donor tissue during storage.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Donor tissue from human or animal sources is widely used for various clinical applications spanning soft and hard tissue repair. Allograft or xenograft transplantation is well established for a variety of use cases, such as skin grafting, tendon repair, structural bone grafts, cartilage defect repair, osteochondral defect repair, bone scaffolding, meniscus replacement, partial joint repair, organ transplants, and more. These tissue graft products are provided sterile or with living tissue preserved with storage technology. Additionally, autologous tissue can be directly harvested from a patient to treat various defects or augment tissue repair. In these instances, senescent cells present may negatively impact that biological properties of the graft and impair integration and healing with the host. The present invention targets removal of the senescent cells during the harvesting and/or storage of the tissue. The targeting and removal of senescent cells varies by cell type so different treatments are tailored for the specific senescent cells. One example of an area impacted by senescent cells is with allograft transplantation.

Allograft Transplantation is well established in the clinical community as a primary treatment option for various conditions such as burn related skin grafts, bone grafts for spinal fusion, or osteochondral allografts (OCAs) for defects spanning a spectrum of patient indications.¹⁻⁴ For example, OCAs have become the clinical standard of care to treat chondral and osteochondral lesions and therefore OCAs are in high clinical demand. The success of this procedure is largely driven by the state of the mature cartilage (e.g., viability, structure) in the OCAs. One of the major limitations of OCAs for healthcare providers and companies that process and/or distribute OCAs is the deterioration of cartilage health during cold storage, which limits both the current shelf-life and can influence patient outcomes clinically.

Several clinical and pre-clinical studies have reported an association between maintaining chondrocyte viability during storage and successful osteochondral repair outcomes.⁵⁻⁹ Moreover, chondrocyte viability in OCAs has consistently been reported to decrease over prolonged storage durations, with a significant loss in chondrocyte viability found after 3-4 weeks of storage⁹⁻¹⁴ leading to an industry standard OCA shelf-life of 28 days.¹⁵⁻¹⁷ However. Increasing OCA donor age has also been found to negatively impact graft survivorship cap of 35 years.¹⁸ Together the requirement for storage times less than 28 days and donor age below 35 years significantly limits OCA tissue availability and creates substantial logistical hurdles for providers and patients.

Chondrocyte death during storage may not be the only deleterious effect on OCA clinical outcomes. Cellular senescence has been shown to be a major contributor to functional deficits in chondrocytes OA.¹⁹⁻²¹ Cellular senescence is a fundamental property of aging defined by loss of proliferative capacity, resistance to apoptosis, and importantly, the release of proinflammatory factors as part of the senescence associated secretory phenotype (SASP).^{22,23} Senescent cells are metabolically active (yet cease to divide) and undergo distinct phenotypic changes including upregulation of p16^{Ink4a} and p21^{Waf1/Cip1} significant secretome changes, telomere shortening, and decompensation of pericentromeric satellite DNA.^{24,25} It is known that the number of senescent cells increases with age in various tissue compartments and an underlying phenomenon associated with numerous age-related pathologies.²⁴⁻²⁶ Importantly, senescent chondrocytes and their cognate SASP factors are known to accumulate with age or after injury and have been found to promote the progression of cartilage deterioration during osteoarthritis (OA)^{27,28} through the secretion of pro-inflammatory cytokines, chemokines, and proteases. In fact, preclinical studies have shown that transplantation of senescent cells induces cartilage decline and OA symptomology²⁹, and genetic or pharmacological elimination of senescent cells specifically in knee joints of mice can alleviate cartilage deterioration and OA symptoms.^{30,31} Considering chondrocyte senescence significantly impairs cartilage health and promotes cartilage decline, we posit the targeted removal of senescent cells in cartilage or bone tissue may improve clinical outcomes, especially in cartilage grafts from older donors.

The residual bone marrow in OCAs primarily consists of fatty bone marrow which contains adipose cells that store lipid components. Therefore, it is plausible that these bioactive lipid metabolites could be released from residual bone marrow and negatively affect chondrocyte health and induce ROS and senescence. Indeed, findings from our group have identified elevated levels of free fatty acids (FFA) and high levels of primary lipid oxidation during prolonged OCA storage that were directly correlated with chondrocyte viability.

Importantly, lipid biosynthetic pathways are dysregulated during senescence and lipid droplet accumulation is a signature of senescent cells versus normal proliferating cells.³²⁻³⁸ FFAs and their oxidized bi-products have also been shown to promote senescence in a variety of cell types including chondrocytes.³⁹ Accordingly, pharmacological inhibition of cysteinyl leukotrienes (LOX mediated oxidized PUFAs) in TNF-α stimulated human primary chondrocytes has been found to down-regulate senescence hallmarks and SASP production with OA.⁴⁰ Therefore, the elevated oxidized lipid components and FFAs found in the storage media may increase senescence in the donor tissue. This invention also describes senolytic compounds that can specifically combat senescence resulting from elevated lipid components and oxidized lipids in the media. Recently, several senolytic compounds that selectively kill senescent cells in vitro and in vivo without affecting quiescent or proliferating cells were identified.^{41,42} While numerous compounds with senolytic properties have been characterized, the drugs can exhibit significant variance in efficacy depending on cell or tissue type.⁴¹⁻⁴³ Efficacy variance exists as not all senescent cells are phenotypically identical. Senescent cells can express different SASP factors, have different senescent markers, or hijack different mechanisms to avoid apoptosis depending on cell type or pathological setting.⁴³⁻⁴⁵ Current strategies suggest the use of multiple combinations of senolytic therapies to maximize efficacy.⁴¹⁻⁴⁵ For example, fisetin is a potent senolytic agent found to target senescence associated pathways such as SIRT1⁴⁶, BCL-2/BCL-X_{L}^{47,48}, HIF-1α⁴⁹, p53/MDM2^{48,50}, and AKT^{48,51} leading to elimination of senescent cells and reduction in SASP driven inflammation in vitro and in vivo. Metformin (a widely prescribed anti-diabetic drug) is another senolytic compound that has been shown to extend lifespan in mice⁵² and exhibits senolytic activity via targeting some similar but also several different senescence associated pathways from fisetin. Metformin is known to activate AMPK which indirectly inhibits mTOR, the most established nutrient sensing longevity regulating pathway to date.⁵³ In addition, metformin reduces oxidative stress and SASP related inflammation through inhibition of NF-KB activity^{53,54} and inhibits insulin and IGF-1 signaling which are all hyperactivated during aging.⁵³⁻⁵⁵ Thus, combinatorial senolytic approaches are preferred if possible. Finally, many senolytic drugs are also chemotherapeutic drugs with potential side effects in vivo. Importantly, the use of multiple senolytics and the avoidance of in vivo side effects is completely allowable to remove senescence cells and detrimental effects of senescence cells from donor and autologous tissue grafts.

To improve donor tissue (e.g. osteochondral allografts or viable bone or dermal grafts) or autologous cell and tissue health our invention describes various formulations of senolytic strategies to reduce oxidative stress, eliminate existing senescent cells, and reduce deleterious effects of oxidized FFAs including induction of cell death or senescence. An example involves treating OCA or bone allograft with viable cells with media and/or storing in media containing additives that target senescence directly or indirectly (example of indirect target involves the reduction of FFAs and their oxidized biproducts) should increase donor age allowances and improve the retention of healthy and functional cells imperative for clinical efficacy.

### BRIEF SUMMARY OF THE INVENTION

According to one aspect of the present disclosure, the invention provides a method of removing senescent cells from donor tissues or cells, organs, or autologous cells or tissues. The tissue graft includes intact minimally manipulated donor tissues as well as tissue engineered grafts. The method comprises of adding at least one senolytic therapy to the tissue or cells during processing or prior to storage.

A senolytic therapy could involve any strategy to reduce senescent cells, reduce senescence phenotype, reduce factors that can cause senescent cells, target known senescence signaling (e.g. inhibitors or RNA therapeutics).

In another aspect, the invention provides a method of enriching the tissue graft, the method comprising adding at least one senolytic therapy to the tissue graft. Example therapies include a senolytic agent that clears senescent cells or senomorphic agent that reduces senescence phenotype, and inhibitors and RNA therapeutics that target senescence signaling.

In certain embodiments of methods according to the invention, the donor or autologous tissues may comprise bone, cartilage, tendon, skin, organs and/or adipose, or placental origins. In additional embodiments, at least 25% of the senescent cells are removed, at least 50% of the senescent cells are removed, at least 75% of the senescent cells are removed, at least 80% of the senescent cells are removed, at least 85% of the senescent cells are removed, at least 90% of the senescent cells are removed, or at least 95% of the senescent cells are removed.

In certain embodiments, the donor or autologous tissue graft contains at least 10% reduction in senescence phenotype (e.g. ROS or SASP production), at least 25% reduction in senescence phenotype, at least 50% reduction in senescence phenotype, at least 75% reduction in senescence phenotype, at least 80% reduction in senescence phenotype, at least 85% reduction in senescence phenotype, at least 90% reduction in senescence phenotype

In certain embodiments of methods according to the invention, the disease, disorder, or condition is tendonitis.

In certain embodiments of methods according to the invention, the disease, disorder, or condition is a tumor.

In certain embodiments of methods according to the invention, the disease, disorder, or condition is cancer.

In certain embodiments of methods according to the invention, the disease, disorder, or condition is osteoarthritis.

In certain embodiments of methods according to the invention, the disease, disorder, or condition is osteochondral defect.

In certain embodiments of methods according to the invention, the disease, disorder, or condition is a tissue void that is created through trauma or surgically created.

In still another aspect, the invention provides a method of improving tissue repair in a subject, the method comprising removing senescent cells from the donor tissue grafts and then implanted into the surgical site. Additionally, the method could involve removing senescent cells from autologous tissue harvested from a patient and implanted into the same patient's recipient site.

In certain embodiments of methods according to the invention, the patient outcome is pain reduction. In additional embodiments of methods according to the invention, the patient outcome is engraftment or graft healing. In additional embodiments of methods according to the invention, the surgical outcome is enhanced functional mobility of the joint or biomechanical properties of the tissues at subsequent follow-up.

In another aspect, the invention provides a method of removing senescent cells from an allogeneic, xenogeneic, or autologous tissue graft, the method comprising adding at least one senolytic agent to the product. The method may involve treatment with at least one SASP inhibitor or a combination of senolytic agents and SASP inhibitors. Additionally, the treatment may involve other senescence targeted therapies like telomerase or drugs that extend telomere length or the treatment with high oxygen. The allograft product may be selected, without limitation, from the group consisting of a bone graft, chondral graft, osteochondral graft, amniotic fluid, placental tissue, and umbilical cord tissue.

In certain embodiments of methods according to the invention, at least one senolytic agent is fisetin, quercetin, 17-DMAG, fenofibrate, dasatinib, UBX0101, navitoclax, ABT-737; or curcumin

In certain embodiments of methods according to the invention, at least one senolytic agent is a BCL-2 family inhibitor.

In certain embodiments of methods according to the invention, at least one senolytic agent is a HSP90 inhibitor.

In certain embodiments of methods according to the invention, at least one senolytic agent targets p53.

In certain embodiments of methods according to the invention, at least one senolytic agent is a cardiac glycoside.

In certain embodiments of methods according to the invention, at least one senolytic agent is a Proteolysis Targeting Chimera (PROTAC).

In certain embodiments of methods according to the invention, at least one senolytic agent is a galactose-modified senolytic pro-drug.

In one aspect, the invention provides a pharmaceutical composition comprising at least one senolytic agent for use in the treatment of donor OCAs prior or during cold storage.

In still another aspect, the invention provides a pharmaceutical composition comprising at least one senolytic agent for use in the improvement of a surgical outcome.

In certain embodiments of compositions according to the invention, at least one senolytic agent is Fisetin.

The preceding summary is provided to facilitate an understanding of some of the innovative features unique to the present disclosure and is not intended to be a full description. A full appreciation of the disclosure can be gained by taking the entire specification, claims, drawings, and abstract as a whole. Additional objects, features and advantages of the invention will become more readily apparent from the following detailed description when taken in conjunction with the drawings wherein like reference numerals refer to corresponding parts in the several views. Other embodiments of the present invention will become apparent from a review of the ensuing detailed description.

### BRIEF DESCRIPTION OF THE FIGURES

FIG. 1 is a flowchart of a process for an allograft or xenograft product being harvested from a donor, processed with senolytic agents and/or SASP inhibitors, and then stored.

FIG. 2 is a flowchart of a process for an allograft or xenograft product being harvested from a donor, processed with a series of senolytic agents and/or SASP inhibitors, and then stored.

FIG. 3 is a flowchart of a process for an allograft or xenograft product being harvested from a donor and then stored in a solution that contain, at least in part, senolytic agents and/or SASP inhibitors.

FIG. 4 is a flowchart of a process for an allograft or xenograft product being harvested from a donor, processed with senolytic agents and/or SASP inhibitors, and then stored in a solution that contain, at least in part, senolytic agents and/or SASP inhibitors.

FIG. 5 is a flowchart of a process for an allograft or xenograft product being harvested from a donor, processed with senolytic agents and/or SASP inhibitors, and then stored, with sample tissue being inspection pre- and post-senescent cell treatment to verify a reduction in senescent cells.

FIG. 6 is a flowchart of a process for an allograft or xenograft product being harvested from a donor, stored, and then processed with senolytic agents and/or SASP inhibitors before implantation.

FIG. 7 is a flowchart of a process for an autologous tissue being harvested from a donor, processed with senolytic agents and/or SASP inhibitors, and then implanted in the same patient.

### DETAILED DESCRIPTION OF THE PREFERRED EMBODIMENTS

The following detailed description should be read with reference to the drawings in which similar elements in different figures are numbered the same. The detailed description and the drawings, which are not necessarily to scale, set forth illustrative and exemplary embodiments and are not intended to limit the scope of the disclosure. Selected features of any illustrative embodiment can be incorporated into an additional embodiment unless clearly stated to the contrary. While the disclosure is amenable to various modifications and alternative forms, specifics thereof have been shown by way of example in the drawings and will be described in detail. Overall, it should be understood, however, that the intention is not to limit aspects of the disclosure to the particular illustrative embodiments described. On the contrary, the intention is to cover all modifications, equivalents, and alternatives falling within the spirit and scope of the disclosure. It is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

In embodiments of the disclosure that specify the selection of “at least one . . . selected from the group consisting of” or simply “selected from the group consisting of”, the use of the conjunction “and” between the final two items of the list following such language indicates that the items in the sequence are alternatives to one another, and that one (or more) of these items is/are selected. It does not mean that each of the items is necessarily selected.

Although any methods and materials similar or equivalent to those described herein can be used in the practice of the present disclosure, the preferred methods and materials are now described. All publications mentioned herein are incorporated herein by reference to describe in their entirety.

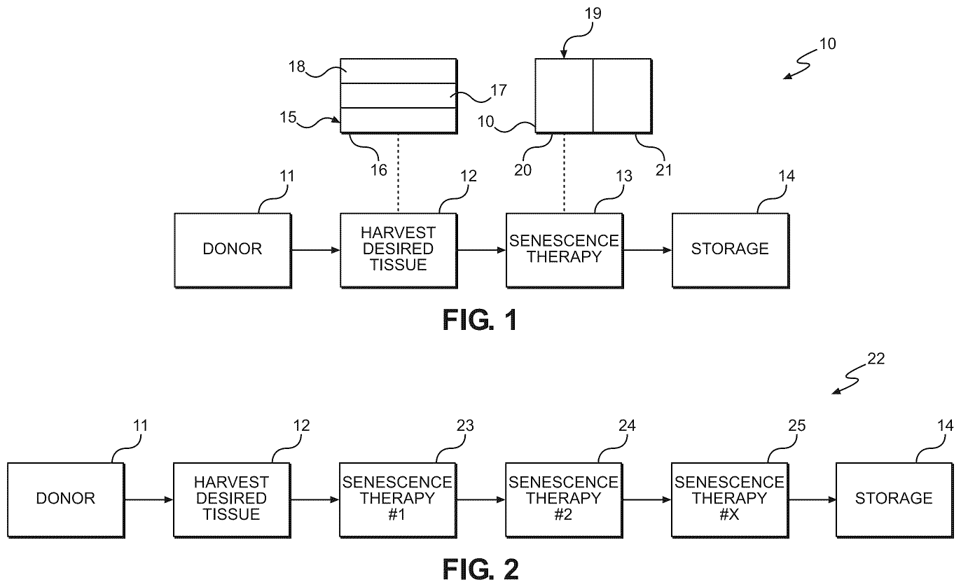

Turning now to FIG. 1 there is shown an example process 10 for forming and processing live tissue grafts. A donor 11 has desired tissue which is harvested at step 12. The tissue undergoes senescence therapy at step 13 and then the tissue is stored at step 14.

The donor 11 could be any person or animal. Preferably donor 11 is a human cadaver. At step 12, a viable tissue graft 15 such as an allograft or xenograft product is harvested from the donor 11. The viable tissue graft 15 includes live tissue 16 with enhanced biological properties. The viable tissue graft 15 includes an amount of senescent cells 17 and an amount of senescence-associated secretory phenotype SASPs 18 distributed throughout the live tissue 16. The live tissue includes tissues from younger donors or older donors that are older than 18 years or tissues impacted with senescence.

The live tissue 16 is preferably allogenic tissue, xenogeneic tissue or engineered tissue and includes bone or osteochondral tissue or soft tissues including a least one of meniscus, cartilage, ligament, tendon, bone, pericardium, intervertebral disc, nervous tissue, blood vessels, heart valves, muscle, dermal tissue, and organs. The live tissue 16 is processed with agents 19 such as senolytic agents 20 and/or SASP inhibitors 21 to reduce the senescent cells 17 and senescence-associated secretory phenotype SASPs 18. The amount of senescent cells 17 is reduced by at least 15% as compared to an amount of senescent cells present in normal tissue and the amount of senescence-associated secretory phenotype SASPs 18 is reduced by 10% as compared to an amount of senescence-associated secretory phenotype SASPs present in normal cells. The senolytic agents 20 include at least one of flavonoids (quercetin, fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof. The SASP inhibitors 21 include at least one of metformin, rapamycin, JAK1/2 inhibitors (e.g., ruxolitinib), and glucocorticoids. The SASP inhibitors include at least one of metformin, rapamycin, JAK1/2 inhibitors (e.g., ruxolitinib), and glucocorticoid.

FIG. 2 shows an example process 22 for the viable tissue graft 15 being harvested from the donor 11 at step 12. The viable tissue graft 15 is processed with a series of processing steps 23, 24 and 25 which employ senolytic agents 20 and/or SASP inhibitors The processing steps preferably include washing the live tissue 16 with the live tissue 16 in aqueous media containing senoltyic agents 20 and/or SASP inhibitors 21 while agitating the media to increase diffusion of the senoltyic agents 19 into the live tissue 16 through sonication or with a bioreactor system. The processing steps 23, 24 and 25 preferably also include soaking the live tissue 16 with the live tissue 16 in aqueous media containing senoltyic agents 19 and/or SASP inhibitors 20.

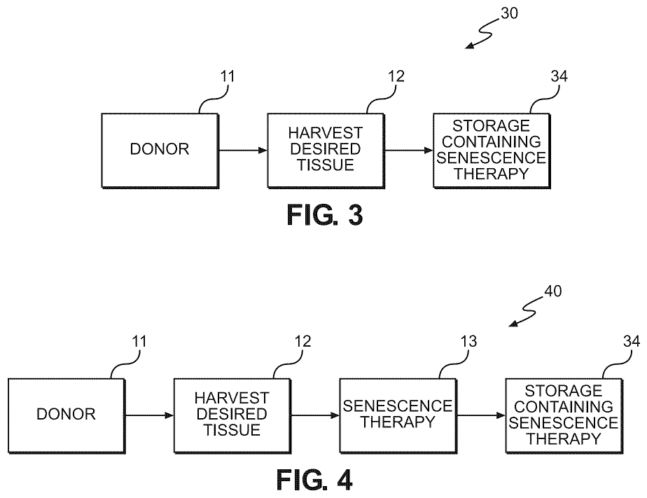

FIG. 3 shows an example process 30 for the viable tissue graft 15 being harvested, at 12, from the donor 11 and then stored in a solution 34 that contains senescence therapy and has, at least in part, senolytic agents 20 and/or SASP inhibitors 21. The viable tissue graft 15 includes senolytic agents or SASP inhibitors to prevent the accumulation of senescent cells 17 during storage 34.

FIG. 4 shows an example process 40 for the viable tissue graft 15 being harvested, at 12, from the donor 11. The viable tissue graft 15 undergoes two therapy steps. The viable tissue graft is processed with senolytic agents and/or SASP inhibitors at step 13, and then stored, at 34, in a solution that contains senescence therapy including, at least in part, senolytic agents 20 and/or SASP inhibitors 21.

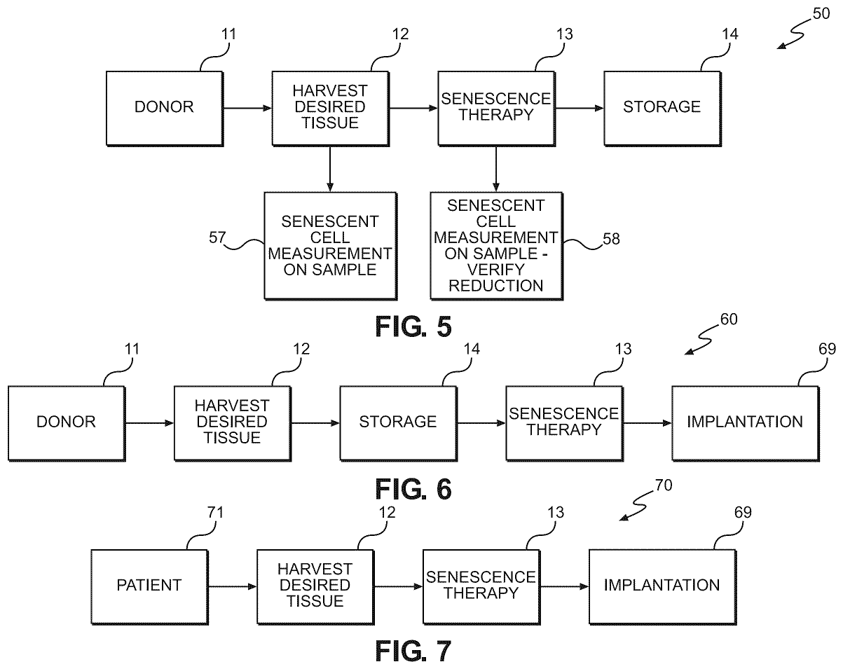

FIG. 5 shows an example process 40 for the viable tissue graft 15 being harvested, at 12, from the donor 11. However, the process 40 includes measurement steps 57 and 58. The viable tissue graft 15, is processed with senolytic agents and/or SASP inhibitors at 13, and then stored at 14. The live tissue 16 is inspected pre-57 and post-58 senescent cell treatment to verify a reduction in senescent cells.

FIG. 6 shows an example process 40 for the viable tissue graft 15 being harvested, at 12, from the donor 11 and adds an implantation step 69 where the viable tissue graft 15 is implanted. More specifically, the viable tissue graft 15 is stored 14, and then processed with senolytic agents and/or SASP inhibitors at step 13, and then stored, at 34 before implantation 69.

FIG. 7 shows an example process 70 for the viable tissue graft 15 being harvested, at 12, from the patient 71. More specifically, the viable tissue graft 15 is stored 14, and then processed with senolytic agents and/or SASP inhibitors at step 13, and then stored, at 34 before implantation 69 of the viable tissue graft 15 back into the same patient 71 who is the donor 71.

### Senescent Cells

Senescent cells include, without limitation, senescent preadipocytes, senescent endothelial cells, senescent fibroblasts, senescent neurons, senescent epithelial cells, senescent chondrocytes, senescent mesenchymal cells, senescent macrophages, senescent bone forming cells (e.g. osteocytes, osteoblasts, osteoclasts), senescent progenitor cells, and senescent smooth muscle cells.

Senescent cells and senescent cell-associated molecules can be detected by techniques and procedures described in the art. For example, the presence of senescent cells in tissues can be analyzed by histochemistry or immunohistochemistry techniques that detect the senescence marker, SA-β galactosidase (SA-β gal).56,57 The presence of the senescent cell-associated polypeptide p16, specifically, p16INK4a and p21Cip1, can be determined by immunochemistry methods practiced in the art, such as immunoblotting analysis.^{58,59} Expression of p16 mRNA in a cell can be measured by techniques practiced in the art including quantitative PCR. The presence and level of senescence cell associated polypeptides (e.g., polypeptides of the SASP, generally called SASP factors or proteins, or senescence messaging secretome (SMS)) can be determined by using automated and high throughput assays.

The presence of senescent cells can also be determined via detection of senescent cell-associated molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject.

### Senolytic Agents

Senolytic agents are agents that selectively target and induce apoptosis/death of senescent cells.43-48 Senolytic agents include, without limitation, flavonoids (quercetin, fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof. Specifically, these agents target senescent cell anti-apoptotic pathways (SCAPs), which are upregulated during senescence. Senolytic agents are sometimes included in a group of interventions known as “geroprotectors” or “senotherapies”. Senolytic agents also include geroprotective nutrients such as, without limitation, myricetin, N-acetyl-cysteine (NAC), gamma tocotrienol, or epigallocatechin-gallate (EGCG). Other terms include “senomorphic” agents which modulate SASP production without direct or specific elimination or apoptosis of senescent cells that are encompassed within these claims.

### Senescence Markers/SASP

Senescent cell markers include, without limitation, increased cell size, accumulation of lipofuscin, high expression of cell cycle regulators (e.g., p16INK4A), p21CIP1, and senescence associated secretory phenotype (SASP) factors (including, without limitation, TNF-alpha, interleukin-6 (IL-6), multifunctional cytokine IL-1beta, chemokines CXCL10, RANTES/CCL5, and MCP-1, matrix metalloprotease MMP3, and serine-protease inhibitor PAI-1 (Senescence Associated Secretory Phenotype (SASP): TNF-alpha, interleukin-6 (IL-6), the multifunctional cytokine IL-1β, the chemokines CXCL10, RANTES/CCL5 and MCP-1, the matrix metalloprotease MMP3, and the serine-protease inhibitor PAI-1⁶⁰⁻⁶² increased cellular senescence-associated β-galactosidase (SA-βgal) activity/accumulation of β-galactosidase, hemostatic factors (e.g., PAI-1), proteases, formation of senescence-associated heterochromatin foci (SAHF), and the appearance of senescent-associated distension of satellites (SADS) and telomere-associated DNA damage foci (TAFs).^{22,43,61} Senescence-associated secretory phenotype (SASP) refers to a phenotype that often develops in senescent cells, marking the dramatic changes in their secretome entails the release of pro-inflammatory cytokines, bradykines, and chemokines, prostanoids, miRNAs, damage-associated molecular pattern proteins (DAMPs), tissue-damaging proteases (i.e., metalloproteases (MMPs)), factors that impact stem and progenitor cell function, hemostatic factors, and growth factors.^{22,43,61,62} SASP factors include, without limitation, interleukins (IL-6, IL-8, IL-1B), monocyte chemoattractant protein-1, and plasminogen-activated inhibitor-1. Senescent cell-associated molecules include those that are described in the art as comprising the senescence-associated secretory phenotype, senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities.^{22,61} Extracellular matrix (ECM)-associated factors include inflammatory proteins and mediators of ECM remodeling that are strongly induced in senescent cells.⁶³ Other senescent cell-associated molecules include extracellular polypeptides (proteins) described collectively as the DNA damage secretory program (DDSP).⁶⁴ Senescent cell-associated proteins also include cell surface proteins (or receptors) that are expressed on senescent cells, which include proteins that are present at a detectably lower amount or are not present on the cell surface of a non-senescent cell. Senescent cell-associated proteins also include cell surface markers, like cluster differentiation markers, that have modified expression (loss or gain) during senescence progression of certain cell types (ex. CD26, CD28).

Senescence cell-associated molecules include secreted factors that may make up the pro-inflammatory phenotype of a senescent cell (e.g., SASP). These factors include, without limitation, GM-CSF, GROα, GROαβγ, IGFBP-7, IL-1γ, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-10, MCP-4, MIF, MIP-3a, MMP-12, MMP-13, MMP-14, NAP2, Oncostatin M, osteoprotegerin, PIGF, RANTES, sgp130, TIMP-2, TRAIL-R3, Acrp30, angiogenin, Axl, bFGF, BLC, BTC, CTACK, EGF-R, Fas, FGF-7, G-CSF, GDNF, HCC-4, I-309, IFN-γ, IGFBP-1, IGFBP-3, IL-1 R1, IL-11, IL-15, IL-2R-a, IL-6 R, I-TAC, Leptin, LIF, MMP-2, MSP-a, PAI-1, PAI-2, PDGF-BB, SCF, SDF-1, sTNF RI, sTNF RII, Thrombopoietin, TIMP-1, tPA, uPA, uPAR, VEGF, MCP-3, IGF-1, TGF-B3, MIP-1-8, IL-4, FGF-7, PDGF-BB, IL-16, BMP-4, MDC, MCP-4, IL-10, TIMP-1, Fit-3 Ligand, ICAM-1, Axl, CNTF, INFY, EGF, BMP-6. Additional identified factors, which include those sometimes referred to in the art as senescence messaging secretome (SMS) factors, some of which are included in the listing of SASP polypeptides, include, without limitation, IGF1, IGF2, and IGF2R, IGFBP3, IDFBP5, IGFBP7, PA11, TGF-β, WNT2, IL-1a, IL-6, IL-8, and CXCR2-binding chemokines. Cell-associated molecules also include, without limitation, the factors described64 and include, for example, products of the genes, MMP1, WNT16B, SFRP2, MMP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAM150B, CST1, GDNF, MUCL1, NPTX2, TMEM155, EDN1, PSG9, ADAMTS3, CD24, PPBP, CXCL3, MMP3, CST2, PSG8, PCOLCE2, PSG7, TNFSF15, C17orf67, CALCA, FGFJ8, IL8, BMP2, MATN3, TFP1, SERPINI 1, TNFRSF25, and IL23A. 22,61,65 Senescent cell-associated proteins also include cell surface proteins (or receptors) that are expressed on senescent cells, which include proteins that are present at a detectably lower amount or are not present on the cell surface of a non-senescent cell.

### SASP Inhibitors

SASP inhibitors are agents that neutralize the senescence-associated secretory phenotype (SASP). SASP inhibitors are sometimes referred to as senostatic or senomorphic agents, which agents modulate a proinflammatory secretome and supplement senolytics for targeting aging and age-related diseases. Senostatic agents selectively suppress the deleterious effects of senescence.⁶⁶ SASP inhibitors include, without limitation, metformin, rapamycin, JAK1/2 inhibitors (e.g., ruxolitinib), and glucocorticoids. In certain embodiments, one or more SASP inhibitors can be used in combination with one or more senolytic agents.

### Treatment Compositions

The present invention includes methods that comprise administering at least one senolytic agent or SASP inhibitor to an allograft, xenograft, donor cells, organs, or autologous cells and tissue, wherein the senolytic agent involves agents that selectively target and induce apoptosis/death of senescent cells. This invention describes the treatment of various living donor or autologous tissues with at least one or multiple senolytic agents. These agents include, without limitation, flavonoids (quercetin, fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof. This invention also describes treatment of the living donor or autologous cells or tissues with at least one SASP inhibitor or a combination of SASP inhibitors, or a combination of senolytic agents and SASP inhibitors.

The present invention includes methods that comprise administering at least one senolytic agent to an allograft or xenograft, wherein the agent is contained within a pharmaceutical composition. The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., a bioengineered scaffold, encapsulation in liposomes, microparticles, microcapsules, recombinant cells capable of expressing the mutant viruses, receptor mediated endocytosis. In a preferred embodiment, the composition is administered as a soluble compound within the tissue storage media. As the aqueous medium for storage, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc.

In certain situations, the pharmaceutical composition can be delivered in a controlled release system. In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138).

In certain situations, the senolytic agents and/or SASP inhibitors may be treat the donor or autologous cells or tissues during processing prior to storage. This may involve harvesting the viable donor tissue and soaking in various senolytic agent(s) and/or SASP inhibitors prior to storage or implantation. The senolytic agents and/or SASP inhibitors may also be applied to the storage media. This tissue process may involve soaking or wash steps with senolytic agents and/or SASP inhibitors. To increase penetration of the agents into tissues or organs sonication, bioreactors, or other methods may be applied to increase diffusion of the agent/inhibitors into the tissue.

### Elimination of Senescent Cells and Tissue Storage and Transplantation

It is desirable to store tissues for as long as possible for future use, including to be given to a recipient for treatment of a condition. It is hypothesized that when tissues are stored, senescent cells are pre-existing (especially with older tissue donors), and senescent cells may accumulate during storage. As a result, the stored tissues implanted into a subject may have a substantial proportion of senescent cells that were native to the donor tissue or induced through the storage.

In one aspect, the disclosure provides methods for clearing senescent cells from tissues in order to minimize the proliferation/accumulation of senescent cells upon (during) graft storage. In one embodiment, senescent cells are cleared from donor tissues prior to storage via treatment with senolytic agents/SASP inhibitors. In still further embodiments, tissues are treated during storage through media that may contain various additives to reduce senescence. In still further embodiments, tissues are treated at the time of surgery, before implantation. In another aspect, the media may be replaced throughout the storage time or used in combination with a bioreactor system. In a preferred embodiment, the tissues are treated during storage with defined storage media containing treatments. By “clearing” senescent cells is meant targeting and killing them. “Clearing” does not necessarily mean that all senescent cells are cleared/killed. Rather, it means that the number of senescent cells in a subject after clearing is measurably lower than beforehand. In some embodiments, at least about 20%, at least about 30%, at least about 40%, at least about 50%, at least about 60%, at least about 70%, at least about 80%, at least about 90%, or at least about 95% of senescent cells are cleared.

In one embodiment, senolytic agents are used to clear senescent cells from tissues that have been stored. Senescent-free (or senescent-reduced) tissues are enriched for implantation into the recipient subject. In one embodiment, purified and enriched senescent-free (or senescent-reduced) tissues are implanted into a recipient site. In one example, the tissue is a living osteochondral allograft that has at least 20% reduction in senescent cells that is implanted into a recipient joint.

### Elimination of Senescent Cells in Allograft or Autologous Products

The term “allograft products”, “xenograft products”, or “osteochondral allograft (OCA)”, as used herein, refers to allogenic products derived from cadaveric donors from tissue banks that are used to improve healing of bone, cartilage, tendon, organs, and/or ligament, for example, after injury or surgery. The products are deemed allogenic because they are procured from a donor to a recipient who is a different person. Allograft products are advantageous in that they minimize the impact of degenerative disease and allow for more rapid recovery from musculoskeletal injury. Xenogeneic products are transplanted from an animal donor to a human. “Autologous products” include harvesting tissue from one location in a patient and implanting the tissue at another site to augment tissue repair in the same patient. This invention also describes the use of senolytic agents intra-operative at the time of autologous graft harvest to clear at least 20% of the senescent cells prior to implanting into the defect or site of implantation.

### Elimination of Senescent Cells in Orthopedic Surgery

Senescent cell burden has been shown to strongly correlate with age-related orthopaedic conditions. The injection of senescent cells is sufficient to drive age-related conditions such as osteoarthritis, frailty, and decreased survival. Thus, the development of therapies that selectively kill senescent cells is anticipated to delay the onset of aging phenotypes, attenuate severity of age-related diseases, improve resiliency, enhance survival, and extend lifespan.^{67,68}

Furthermore, targeting and eliminating senescent cells has been shown to mitigate age-related musculoskeletal decline. Thus, in one aspect, the invention involves the treatment of tissues with senolytic agents to eliminate pre-existing senescent cells in the graft. The tissue is treated with, for example, Fisetin or Quercetin. In another embodiment, the tissue is stored in the presence of senolytic or senomorphic agents or SASP inhibitors, or combinations as a component of the storage media or storage protocol.

An improvement in a surgical outcome means a positive change from baseline. In this context, the term “positive” refers to a change associated with better healing or other clinical outcomes such as improved pain scores or mobility. As used herein, the term “baseline” means the numerical value of the parameter for a subject prior to or at the time of treatment according to the present invention.

To determine whether the surgical outcome/parameter has “improved,” the parameter is quantified at baseline and at one or more time-points after treatment. The difference between the value of the parameter at a particular time point following initiation of treatment and the value of the parameter at baseline is used to establish whether there has been an “improvement”.

### Senescent Cell Detection

To verify a reduction in senescent cells in a tissue graft product, the process or product requires inspection. These methods can include cellular interrogation via flow cytometry and senescence staining, cell expression analysis via flow cytometry, qPCR, and immunofluorescence (IF). Direct senescence detection using β-gal staining and SASP factor analysis of media using immunobeads and flow cytometry.

Many current methods are timely and expensive, which can make them prohibitive tissue bank companies. One aspect of the present invention includes a fluorescent β-gal staining process that is able to be used quicker and more readily on tissues with a fluorescent microscope or other imaging device. This invention involves the development of a quality control measure to confirm reduction in senescent cells or related factors. One example test includes the staining of fluorescent B-gal directly on the tissue graft and evaluation using fluorescent microscopy.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The following citations are incorporated herein by reference.
1. Görtz, S. et al. Metrics of OsteoChondral Allografts (MOCA) Group Consensus Statements on the Use of Viable Osteochondral Allograft. Orthop J Sports Med 9, 1-12 (2021).
2. Familiari, F. et al. Clinical Outcomes and Failure Rates of Osteochondral Allograft Transplantation in the Knee: A Systematic Review. American Journal of Sports Medicine 46, 3541-3549 (2018).
3. Tírico, L. E. P., McCauley, J. C., Pulido, P. A., Demange, M. K. & Bugbee, W. D. Is Patient Satisfaction Associated With Clinical Outcomes After Osteochondral Allograft Transplantation in the Knee? American Journal of Sports Medicine 47, 82-87 (2019).
4. Wang, D. et al. Clinically Meaningful Improvement After Treatment of Cartilage Defects of the Knee With Osteochondral Grafts. American Journal of Sports Medicine 47, 71-81 (2019).
5. Pallante, A. L. et al. The in vivo performance of osteochondral allografts in the goat is diminished with extended storage and decreased cartilage cellularity. American Journal of Sports Medicine 40, 1814-1823 (2012).
6. Pallante, A. L. et al. Treatment of articular cartilage defects in the goat with frozen versus fresh osteochondral allografts: Effects on cartilage stiffness, zonal composition, and structure at six months. Journal of Bone and Joint Surgery-Series A 94, 1984-1995 (2012).
7. Schmidt, K. J., Tírico, L. E., McCauley, J. C. & Bugbee, W. D. Fresh Osteochondral Allograft Transplantation: Is Graft Storage Time Associated with Clinical Outcomes and Graft Survivorship? American Journal of Sports Medicine 45, 2260-2266 (2017).
8. Nuelle, C. W., Nuelle, J. A. V., Cook, J. L. & Stannard, J. P. Patient Factors, Donor Age, and Graft Storage Duration Affect Osteochondral Allograft Outcomes in Knees with or without Comorbidities. Journal of Knee Surgery 30, 179-184 (2017).
9. Tabbaa, S. M., Guilak, F., Sah, R. L. & Bugbee, W. D. Fresh Osteochondral and Chondral Allograft Preservation and Storage Media: A Systematic Review of the Literature. American Journal of Sports Medicine 1-15 (2021) doi: 10.1177/03635465211016832.
10. Pennock, A. et al. Prolonged Storage of Osteochondral Allografts-Does the Addition of Fetal Bovine Serum Improve Chondrocyte Viability?. Journal of Knee Surgery 19, 265-272 (2010).
11. Pallante, A. L. et al. Chondrocyte Viability is Higher after Prolonged Storage at 37° C. Than at 4 C for Osteochondral Grafts. American Journal of Sports Medicine 37, 24S-32S (2009).
12. Pennock, A. T. et al. Does subchondral bone affect the fate of osteochondral allografts during storage? American Journal of Sports Medicine 34, 586-591 (2006).
13. Stoker, A., Garrity, J. T., Hung, C. T., Stannard, J. P. & Cook, J. Improved preservation of fresh osteochondral allografts for clinical use. J Knee Surg 25, 117-125 (2012).
14. Williams, J. M. et al. Prolonged-fresh preservation of intact whole canine femoral condyles for the potential use as osteochondral allografts. Journal of Orthopaedic Research 23, 831-837 (2005).
15. Merkely, G. et al. Shorter Storage Time Is Strongly Associated With Improved Graft Survivorship at 5 Years After Osteochondral Allograft Transplantation. Am J Sports Med 48, 3170-3176 (2020).
16. McAllister, D. R., Joyce, M. J., Mann, B. J. & Vangsness, C. T. Allograft update: the current status of tissue regulation, procurement, processing, and sterilization. Am J Sports Med 35, 2148-58 (2007).
17. Dontchos, B. N. et al. Optimizing CO2 normalizes pH and enhances chondrocyte viability during cold storage. J Orthop Res 26, 643-50 (2008).
18. Tabbaa, S. M., Guilak, F., Sah, R. L. & Bugbee, W. D. Fresh Osteochondral and Chondral Allograft Preservation and Storage Media: A Systematic Review of the Literature. Am J Sports Med 50, 1702-1716 (2022).
19. Jeon, O. H., David, N., Campisi, J. & Elisseeff, J. H. Senescent cells and osteoarthritis: a painful connection. J Clin Invest 128, 1229-1237 (2018).
20. Martin, J. A. & Buckwalter, J. A. Telomere erosion and senescence in human articular cartilage chondrocytes. J Gerontol A Biol Sci Med Sci 56, B172-9 (2001).
21. Martin, J. A. & Buckwalter, J. A. Roles of articular cartilage aging and chondrocyte senescence in the pathogenesis of osteoarthritis. Iowa Orthop J 21, 1-7 (2001).
22. Coppé, J.-P., Desprez, P.-Y., Krtolica, A. & Campisi, J. The senescence-associated secretory phenotype: the dark side of tumor suppression. Annu Rev Pathol 5, 99-118 (2010).
23. Cuollo, L., Antonangeli, F., Santoni, A. & Soriani, A. The Senescence-Associated Secretory Phenotype (SASP) in the Challenging Future of Cancer Therapy and Age-Related Diseases. Biology (Basel) 9, (2020).
24. Kumari, R. & Jat, P. Mechanisms of Cellular Senescence: Cell Cycle Arrest and Senescence Associated Secretory Phenotype. Front Cell Dev Biol 9, (2021).
25. Huang, W., Hickson, L. J., Eirin, A., Kirkland, J. L. & Lerman, L. O. Cellular senescence: the good, the bad and the unknown. Nat Rev Nephrol 18, 611-627 (2022).
26. Zhang, L. et al. Cellular senescence: a key therapeutic target in aging and diseases. J Clin Invest 132, (2022).
27. Coryell, P. R., Diekman, B. O. & Loeser, R. F. Mechanisms and therapeutic implications of cellular senescence in osteoarthritis. Nat Rev Rheumatol 17, 47-57 (2021).
28. Liu, Y., Zhang, Z., Li, T., Xu, H. & Zhang, H. Senescence in osteoarthritis: from mechanism to potential treatment. Arthritis Res Ther 24, 174 (2022).
29. Xu, M. et al. Transplanted Senescent Cells Induce an Osteoarthritis-Like Condition in Mice. J Gerontol A Biol Sci Med Sci glw154 (2016) doi: 10.1093/gerona/glw154.
30. Peilin, W. et al. Directed elimination of senescent cells attenuates development of osteoarthritis by inhibition of c-IAP and XIAP. Biochimica et Biophysica Acta (BBA)-Molecular Basis of Disease 1865, 2618-2632 (2019).
31. Jeon, O. H. et al. Local clearance of senescent cells attenuates the development of post-traumatic osteoarthritis and creates a pro-regenerative environment. Nat Med 23, 775-781 (2017).
32. Chee, W.-Y. et al. B-catenin-promoted cholesterol metabolism protects against cellular senescence in naked mole-rat cells. Commun Biol 4, 357 (2021).
33. Lizardo, D. Y., Lin, Y.-L., Gokcumen, O. & Atilla-Gokcumen, G. E. Regulation of lipids is central to replicative senescence. Mol Biosyst 13, 498-509 (2017).
34. Flor, A. C., Wolfgeher, D., Wu, D. & Kron, S. J. A signature of enhanced lipid metabolism, lipid peroxidation and aldehyde stress in therapy-induced senescence. Cell Death Discov 3, 17075 (2017).
35. Cox, L. S. & Redman, C. The role of cellular senescence in ageing of the placenta. Placenta 52, 139-145 (2017).
36. Ogrodnik, M., Salmonowicz, H. & Gladyshev, V. N. Integrating cellular senescence with the concept of damage accumulation in aging: Relevance for clearance of senescent cells. Aging Cell 18, (2019).
37. Hamsanathan, S. & Gurkar, A. U. Lipids as Regulators of Cellular Senescence. Front Physiol 13, (2022).
38. Ogrodnik, M. et al. Cellular senescence drives age-dependent hepatic steatosis. Nat Commun 8, 15691 (2017).
39. Hamsanathan, S. & Gurkar, A. U. Lipids as Regulators of Cellular Senescence. Front Physiol 13, 796850 (2022).
40. Song, W. et al. Antagonism of cysteinyl leukotriene receptor 1 (cysLTR1) by montelukast suppresses cell senescence of chondrocytes. Cytokine 103, 83-89 (2018).
41. van Deursen, J. M. Senolytic therapies for healthy longevity. Science 364, 636-637 (2019).
42. Kirkland, J. L., Tchkonia, T., Zhu, Y., Niedernhofer, L. J. & Robbins, P. D. The Clinical Potential of Senolytic Drugs. J Am Geriatr Soc 65, 2297-2301 (2017).
43. Kirkland, J. L. & Tchkonia, T. Cellular Senescence: A Translational Perspective. EBioMedicine 21, 21-28 (2017).
44. Niedernhofer, L. J. & Robbins, P. D. Senotherapeutics for healthy ageing. Nat Rev Drug Discov 17, 377-377 (2018).
45. Zhu, Y. et al. The Achilles' heel of senescent cells: from transcriptome to senolytic drugs. Aging Cell 14, 644-658 (2015).
46. Zheng, W. et al. Fisetin inhibits IL-1ß-induced inflammatory response in human osteoarthritis chondrocytes through activating SIRT1 and attenuates the progression of osteoarthritis in mice. Int Immunopharmacol 45, 135-147 (2017).
47. Pal, H. C., Sharma, S., Elmets, C. A., Athar, M. & Afaq, F. Fisetin inhibits growth, induces G2/M arrest and apoptosis of human epidermoid carcinoma A 431 cells: role of mitochondrial membrane potential disruption and consequent caspases activation. Exp Dermatol 22, 470-475 (2013).
48. Zhu, Y. et al. New agents that target senescent cells: the flavone, fisetin, and the BCL-XL inhibitors, A1331852 and A1155463. Aging 9, 955-963 (2017).
49. Triantafyllou, A., Mylonis, I., Simos, G., Bonanou, S. & Tsakalof, A. Flavonoids induce HIF-1α but impair its nuclear accumulation and activity. Free Radic Biol Med 44, 657-670 (2008).
50. Li, J. et al. Fisetin, a Dietary Flavonoid, Induces Cell Cycle Arrest and Apoptosis through Activation of p53 and Inhibition of NF-Kappa B Pathways in Bladder Cancer Cells. Basic Clin Pharmacol Toxicol 108, 84-93 (2011).
51. Zhang, X.-J. & Jia, S.-S. Fisetin inhibits laryngeal carcinoma through regulation of AKT/NF-KB/mTOR and ERK1/2 signaling pathways. Biomedicine & Pharmacotherapy 83, 1164-1174 (2016).
52. Martin-Montalvo, A. et al. Metformin improves healthspan and lifespan in mice. Nat Commun 4, 2192 (2013).
53. Barzilai, N., Crandall, J. P., Kritchevsky, S. B. & Espeland, M. A. Metformin as a Tool to Target Aging. Cell Metab 23, 1060-1065 (2016).
54. Moiseeva, O. et al. Metformin inhibits the senescence-associated secretory phenotype by interfering with KK/NF-KB activation. Aging Cell 12, 489-498 (2013).
55. Liu, B. et al. Potent anti-proliferative effects of metformin on trastuzumab-resistant breast cancer cells via inhibition of erbB2/IGF-1 receptor interactions. Cell Cycle 10, 2959-2966 (2011).
56. Lee, B. Y. et al. Senescence-associated β-galactosidase is lysosomal β-galactosidase. Aging Cell 5, 187-195 (2006).
57. Dimri, G. P. et al. A biomarker that identifies senescent human cells in culture and in aging skin in vivo. Proc Natl Acad Sci USA 92, 9363-7 (1995).
58. Idda, M. L. et al. Survey of senescent cell markers with age in human tissues. Aging 12, 4052-4066 (2020).
59. Giatromanolaki, A., Kouroupi, M., Balaska, K. & Koukourakis, M. I. Immunohistochemical detection of senescence markers in human sarcomas. Pathol Res Pract 216, 152800 (2020).
60. Rao, S. G. & Jackson, J. G. SASP: Tumor Suppressor or Promoter? Yes! Trends Cancer 2, 676-687 (2016).
61. Sun, Y., Coppé, J.-P. & Lam, E. W.-F. Cellular Senescence: The Sought or the Unwanted? Trends Mol Med 24, 871-885 (2018).
62. Tchkonia, T., Zhu, Y., van Deursen, J., Campisi, J. & Kirkland, J. L. Cellular senescence and the senescent secretory phenotype: therapeutic opportunities. Journal of Clinical Investigation 123, 966-972 (2013).
63. Kuilman, T. & Peeper, D. S. Senescence-messaging secretome: SMS-ing cellular stress. Nat Rev Cancer 9, 81-94 (2009).
64. Sun, Y. et al. Treatment-induced damage to the tumor microenvironment promotes prostate cancer therapy resistance through WNT16B. Nat Med 18, 1359-1368 (2012).
65. Young, A. R. J. & Narita, M. SASP reflects senescence. EMBO Rep 10, 228-30 (2009).
66. Kang, C. Senolytics and Senostatics: A Two-Pronged Approach to Target Cellular Senescence for Delaying Aging and Age-Related Diseases. Mol Cells 42, 821-827 (2019).
67. Xu, M. et al. Transplanted Senescent Cells Induce an Osteoarthritis-Like Condition in Mice. J Gerontol A Biol Sci Med Sci glw154 (2016) doi: 10.1093/gerona/glw154. Xu, M. et al. Senolytics improve physical function and increase lifespan in old age. 68. Nat Med 24, 1246-1256 (2018).

1. A viable tissue graft comprising:
live tissue with enhanced biological properties, the live tissue including an amount of senescent cells and an amount of senescence-associated secretory phenotype SASPs,
wherein the amount of senescent cells is reduced by at least 15% as compared to an amount of senescent cells present in normal tissue and
wherein the amount of senescence-associated secretory phenotype SASPs is reduced by at least 10% as compared to an amount of senescence-associated secretory phenotype SASPs present in normal cells.

2. The viable tissue graft of claim 1, wherein the live tissue including at least one of allogenic tissue, xenogeneic tissue and engineered tissue.

3. The viable tissue graft of claim 2, wherein the live tissue includes:
bone or osteochondral tissue or
soft tissues including a least one of meniscus, cartilage, ligament, tendon, bone, pericardium, intervertebral disc, nervous tissue, blood vessels, heart valves, muscle, dermal tissue, and organs.

4. The viable tissue graft of claim 2, further comprising senolytic agents or SASP inhibitors to prevent accumulation of senescent cells during storage.

5. The viable tissue graft of claim 4, wherein the senolytic agents include at least one of flavonoids (quercetin, fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof.

6. The viable tissue graft of claim 4, wherein where the SASP inhibitors include at least one of metformin, rapamycin, JAK1/2 inhibitors (e.g., ruxolitinib), and glucocorticoids.

7. The viable tissue graft of claim 3, wherein the live tissue includes tissues from donors that are older than 18 years or tissues impacted with senescence.

8. A method for forming and processing live tissue grafts with enhanced biological properties comprising:
obtaining a tissue with an amount of senescent cells and
reducing the amount of senescent cells and SASPs in the tissue by applying senolytic agents or SASP inhibitors.

9. The method according to claim 8, wherein applying senolytic agents and/or SASP inhibitors includes applying senolytic agents including at least one of flavonoids (quercetin, fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof.

10. The method according to claim 8, wherein applying senolytic agents or SASP inhibitors includes applying SASP inhibitors including at least one of metformin, rapamycin, JAK1/2 inhibitors, ruxolitinib, and glucocorticoids.

11. The method according to claim 8, further comprising washing the live tissue with the live tissue in aqueous media containing senoltyic agents and/or SASP inhibitors while agitating the media to increase diffusion of the senoltyic agents into the live tissue through sonication or with a bioreactor system.

12. The method according to claim 8, further comprising soaking the live tissue with the live tissue in aqueous media containing senoltyic agents and/or SASP inhibitors.

13. The method according to claim 8, further comprising storing the tissue graft in an aqueous media with senoltyic agent(s) and/or SASP inhibitors to mitigate accumulation of senescence during storage.

14. The method according to claim 8, further comprising treating the tissue graft prior to storage with senoltyic agents or SASP inhibitors during storage to reduce senescent cells prior to storage and prevent development of senescence during storage.

15. The method according to claim 8, further comprising treating the tissue graft with an aqueous media with senoltyic agent(s) and/or SASP inhibitors after being removed from storage, prior to implantation.
